(19) United States
(12) Patent Application Publication
(10) Pub. No.: US 2013/0023904 A1
(43) Pub. Date: Jan. 24, 2013

(54) Suturing method

(71) Applicants: Yoshinori MORITA, Kobe-shi (JP); Shinji TAKAHASHI, Tokyo (JP); Tetsuya YAMAMOTO, Hanno-shi (JP); Takayuki SUZUKI, Yokohama-shi (JP)

(72) Inventors: Yoshinori MORITA, Kobe-shi (JP); Shinji TAKAHASHI, Tokyo (JP); Tetsuya YAMAMOTO, Hanno-shi (JP); Takayuki SUZUKI, Yokohama-shi (JP)

(73) Assignees: Yoshinori Morita, Hyogo-ken (JP); OLYMPUS MEDICAL SYSTEMS CORP., Tokyo (JP)

(21) Appl. No.: 13/186,788

(22) Filed: Jul. 20, 2011

(51) Int. Cl. A61B 17/04

(52) U.S. Cl. 606/144

(57) ABSTRACT

A suturing method includes inserting an endoscope having a pressing part attached to a distal end thereof into a body cavity, and moving the endoscope to a near side of tissue; inserting a suturing instrument having a suturing unit mounted thereon into a channel of the endoscope, and projecting a distal part from the distal end of the endoscope; moving the anchor holder having the plurality of anchors accommodated therein to a back side opposite to the near side of the tissue; and inserting the distal end of the anchor holder into the tissue from the back side opposite to the near side while the tissue is pressed by the pressing part from the near side, and ejecting at least one of the plurality of anchors from the anchor holder which is attached to ends of a suture thread in the suturing unit.

### BACKGROUND OF THE INVENTION

1. Field of the Invention

The present invention relates to a suturing method, and more particularly, to a suturing method using a suturing apparatus that sutures a perforation OT the like formed in the tissue of hollow organs, such as the stomach and intestines, using a suturing unit in which anchors are attached to the ends of a suture thread.

2. Background Art

In the related art, a suturing instrument using a suture thread that has anchors attached to both ends thereof in order to suture a perforation, a laceration, or the like that is formed in the tissue of hollow organs, such as the stomach and intestines, is known (for example, refer to International Publication No. 2007-37326). In this suturing instrument, suturing is performed by locking the anchors attached to both ends of the suture thread around a perforation in the tissue or outside of the tissue, and pulling and tightening the suture thread to draw the tissue having the anchors locked thereon.

Moreover, International Publication No. 2007-37335 describes that the suturing instrument of the same system includes a base having flexibility, an anchor holder that is arranged at a distal end of the base, is capable of accommodating a plurality of anchors therein, and has a distal end directed to a proximal end of the base, and a distal end position changing mechanism that moves the anchor holder in a direction that crosses an axis of the base in a length direction thereof. In this suturing instrument, if an attraction jig is brought close to the anchor, the anchor is released by magnetism from the anchor holder.

### SUMMARY OF THE INVENTION

The present invention provides a suturing method of tissue using a suture apparatus including a suturing instrument that has a hollow anchor holder at a distal part thereof, the hollow anchor holder having a sharp distal end and having a plurality of anchors accommodated in an inner cavity thereof, and that performs suturing of tissue using a suture unit in which the plurality of anchors is attached to ends of a suture thread; an endoscope including an insertion part having a channel that allows the suturing instrument to be inserted thereinto; and a pressing part capable of being mounted on a distal end of the endoscope. The suturing method includes inserting the endoscope having the pressing part attached to the distal end thereof into a body cavity, and moving the endoscope to a near side of the tissue; inserting the suturing instrument having the suture unit mounted thereon into the channel, and projecting the distal part from the distal end of the endoscope; moving the anchor holder having the plurality of anchors accommodated therein to a back side opposite to the near side of the tissue; and inserting the distal end of the anchor holder into the tissue from the back side opposite to the near side while the tissue is pressed by the pressing part from the near side, and ejecting at least one of the plurality of anchors from the anchor holder.

### BRIEF DESCRIPTION OF THE DRAWINGS

FIG. 1 is a view showing a suturing instrument used for a suturing method of a first embodiment of the invention.

FIG. 2 is an enlarged partially cross-sectional view showing a distal part of the suturing instrument.

FIG. 3 is a view showing a suturing unit used for the suturing instrument.

FIG. 4 is a view when a needle and a pusher part of the suturing instrument are seen from a pulling portion side.

FIG. 5 is an enlarged partially cross-sectional view showing an operating part of the suturing instrument.

FIGS. 6 to 17 are views showing an operation when the suturing instrument is used.

FIG. 18 is a view showing a distal part in a modification of the suturing instrument.

FIG. 19 is a view showing a distal part in a modification of the suturing instrument.

FIG. 20 is a view showing a distal part in a suturing instrument used for a suturing method of a second embodiment of the invention.

FIG. 21 is a view showing a distal part in a modification of a suturing apparatus of the invention.

FIG. 22 is a view showing a cap in a modification of the suturing apparatus of the invention.

FIG. 23 is a view showing a distal part in a modification of the suturing apparatus of the invention.

FIGS. 24 to 26 are views showing an operation when the suturing apparatus is used.

FIG. 27 is a view showing a cap in a modification of the suturing apparatus related to the invention.

FIG. 28 is a view showing an operation when the suturing apparatus is used.

### PREFERRED EMBODIMENTS

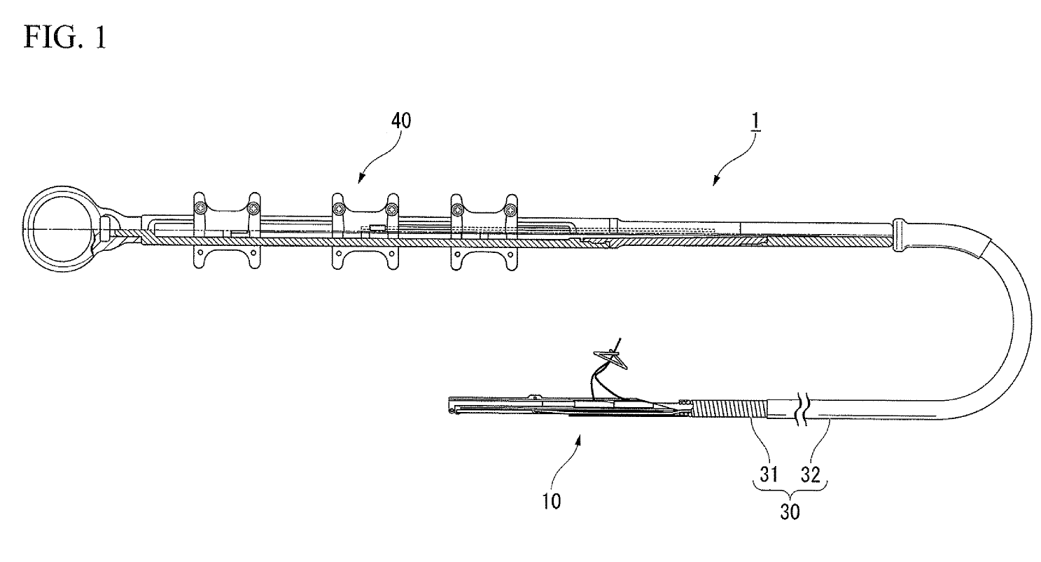

A suturing method of a first embodiment of the invention will be described with reference to FIGS. 1 to 19.

FIG. 1 is a view showing a suturing instrument 1 used for the suturing method of the present embodiment. As shown in FIG. 1, the suturing instrument 1 is adapted to include a distal part 10 inserted into the inside of the body, and an operating part 40 for operating mechanisms of the distal part 10.

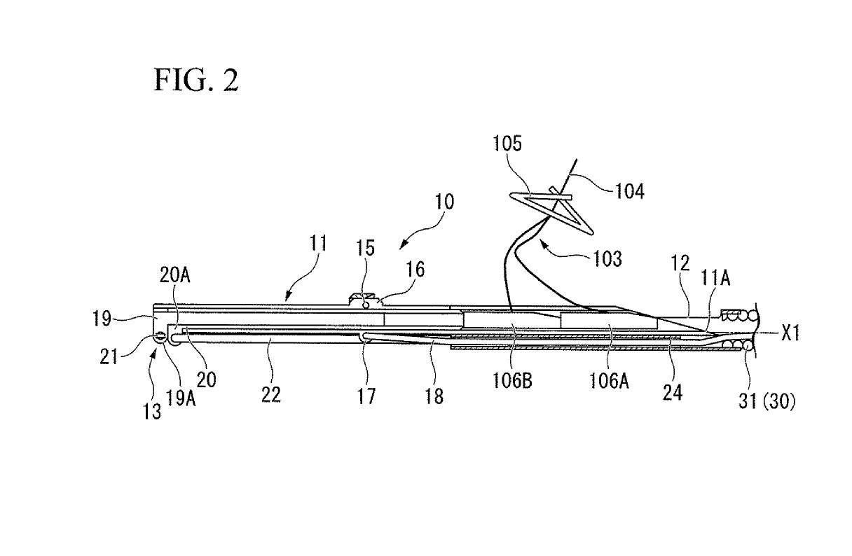

FIG. 2 is an enlarged partially cross-sectional view showing the distal part 10. The distal part 10 includes a needle (anchor holder) 11 to which a suturing unit that will be described below is attached, a base 12 to which the needle 11 is turnably attached, and a pusher part 13 for ejecting the suturing unit.

The needle 11 is a hollow member formed of metal or the like, and is obliquely cut so that a distal end 11A forms an acute angle with an axis. An anchor of a suturing unit 103 is accommodated in an inner cavity of the needle 11 from a sharp distal end 11A thereof.

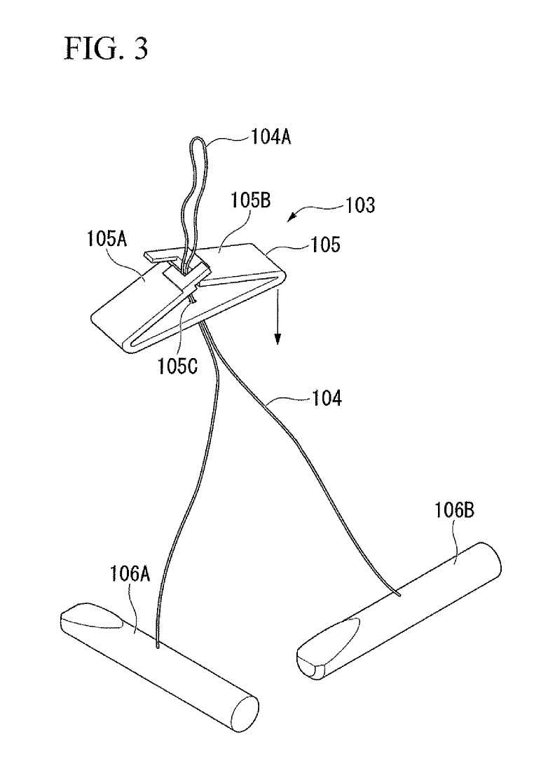

FIG. 3 is a view showing the suturing unit 103 accommodated in the needle 11. The suturing unit 103 has a well-known configuration as described in, for example, US Patent Application Laid-Open Publication No. 2009-0259232, and includes a suture thread 104, a stopper 105 through which the suture thread 104 is inserted, and a first rod-shaped anchor 106A and a second rod-shaped anchor 106B that are attached to both ends of the suture thread 104.

The stopper 105 is bent such that right and left ends 105A and 105B of a plate-shaped member made of metal, resin, such as biodegradable resin, or the like face each other, and is formed such that the ends 105A and 105B are engaged with each other.

A hole 105C is provided near the center of the stopper 105 in a right-and-left direction thereof, and the suture thread 104 that is bent at a middle point 104A is arranged so as to be inserted through the hole 105C from a surface opposite to the ends 105A and 105B and pass between the ends 105A and 105B that engage each other.

The first anchor 106A and the second anchor 106B are substantially round-bar-shaped members, and have the suture thread 104 connected to positions near the longitudinal centers thereof. Each of the anchors 106A and 106B is cut so as to form an acute angle with respect to each axis.

As shown in the partially cross-sectional view of FIG. 2, the first anchor 106A and the second anchor 106B are accommodated in the distal end 11A of the needle 11 such that the first anchor 106A is located closer to the distal side. The suture thread 104 and the stopper 105 that are connected to the anchors 106A and 1068, respectively, are exposed to the outside of the needle 11. In addition, the suturing unit of the invention is not limited to that of the present embodiment, and for example, one, or three or more anchors 106 may be provided, or a well-known thread stop member that is a separate member (not shown) may be used after suturing without providing the stopper 105.

The base 12 is a member that is made of resin, a metal, or the like, and is formed in a substantially tubular shape. A turning shaft 15 that forms a right angle with an axis X1 of the tubular base 12 is attached to a distal portion of the base 12, and a proximal side of the base is connected to a sheath part 30 that connects the distal part 10 and the operating part 40. The sheath part 30, as shown in FIG. 1, is made up of an inside coil sheath 31, and a resin sheath 32 through which the coil sheath 31 is inserted in a manner capable of advancing or retreating.

The part of an outer peripheral surface of the base 12 that is located in the same phase as the turning shaft 15 is cut out over a predetermined range in a longitudinal direction so as not to interfere with the rotation of the needle 11. Additionally, although the outer peripheral surface of the base in a phase that faces the turning shaft 15 across the axis X1 is also cut out over a predetermined range from the distal side, the outer peripheral surface in the phase is left behind to such a degree that the distal end 11A of the needle 11 can be supported.

As shown in FIG. 2, a pivot portion 16 is provided through which the turning shaft 15 is installed on an outer peripheral surface in the same phase as the part of a slope of the distal end 11A closest to the proximal side, at a longitudinal intermediate portion of the needle 11. A pulling portion 17 to which a pulling wire 18 that pulls the needle 11 is connected is formed on the outer peripheral surface of the part that faces the pivot portion 16 across the axis of the needle 11.

The needle 11 is pivoted by the base 12 as the turning shaft 15 is inserted through the pivot portion 16, with the distal end 11A directed to the proximal side of the base 12. Thereby, the posture of the needle 11 can be switched to a first state and a second state by operating to advance or retreat the pulling wire 18 in the axis direction of the base 12. The first state is a state where the portion that is substantially parallel to the base 12 and closer to the distal end 11A side than the pivot portion 16 is accommodated in the inner cavity of the base 12, and the second state is a state where the axis of the needle 11 forms an angle with the axis X1, and the distal end 11A is directed to the direction away from axis X1.

The pusher part 13 includes a first pusher 19 for ejecting the first anchor 106A, and a second pusher 20 for ejecting the second anchor 106B.

The first pusher 19 is a round-bar-shaped member, and one end (a second end) thereof is formed with a connecting portion 19A that protrudes radially outward. A first wire (pusher wire) 21 for operating the first pusher 19 is locked to the connecting portion 19A. The second pusher 20 is a tubular member that has an inner cavity into which the first pusher 19 can be inserted, and has a connecting portion 20A that protrudes radially outward at one end thereof. A second wire (pusher wire) 22 for operating the second pusher 20 is locked to the connecting portion 20A.

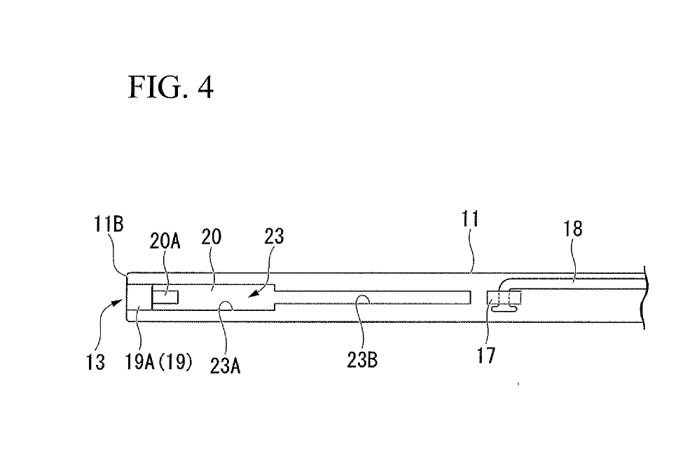

FIG. 4 is a view when the needle 11 and the pusher part 13 are seen from the pulling portion 17 side. In addition, in order to make the drawing easily understood, the drawing is shown excluding the first wire 21 and the second wire 22.

In the needle 11, a groove 23 is formed in the outer peripheral surface of almost the same phase as the pulling portion 17 from the proximal end 11B side of the needle 11. The groove 23 has a first region 23A near the proximal end 11B, and a second region 23B that has a width narrower than the first region 23A and is continuous with the first region 23A. Both the lengths of the first region 23A and the second region 23B are almost equal to the longitudinal length of each of the anchors 106A and 106B of the suturing unit 103.

In the first pusher 19 and the second pusher 20, with the end of the first pusher where the connecting portion 19A is not provided being inserted into the end (the second end) of the second pusher 20 where the connecting portion 20A is provided, the end (the first end) of the second pusher where the connecting portion 20A is not provided is inserted into the proximal end 11B of the needle 11. The respective connecting portions 19A and 20A are arranged such that the phases thereof in a circumferential direction are matched with the groove 23, and as the connecting portions 19A and 20A move in the groove 23, the pusher part 13 can advance or retreat in the axis direction with respect to the needle 11. In the groove 23, the dimensions of the groove 23 and the respective connecting portions 19A and 20A are set so that, as for the width of the first region 23A, both of the connecting portions 19A and 20A can advance into the first region, and as for the width of the second region 23B, only the connecting portion 20A can advance into the second region.

The wires 21 and 22 connected to the pulling wire 18 and the pusher part 13, as shown in FIG. 2, extend up to the operating part 40 through the inside of the coil sheath 31. These three wires extend into the coil sheath 31 through a guide 24 provided in the base 12 so as not to cause meandering or buckling in the inner cavity of the base 12.

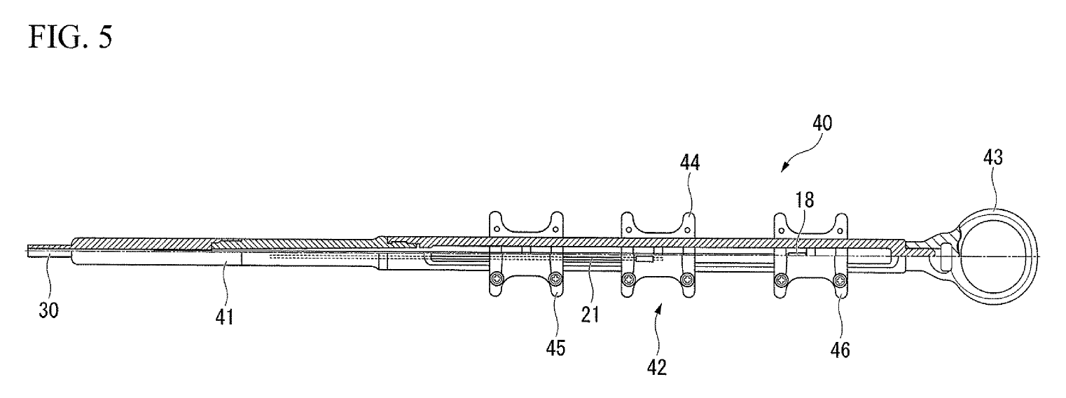

FIG. 5 is a partially cross-sectional view showing the operating part 40. The operating part 40 includes a main body 41 to which a proximal end of the coil sheath 31 is fixed, and a sliding portion 42 that is attached to the main body 41 so as to be slidable in the axis direction of the main body 41.

The main body 41 is formed of resin or the like, and has therein a space along which the above-described three wires pass. Although the proximal end of the coil sheath 31 is connected to a distal portion of the main body 41, the proximal end of the resin sheath 32 can advance or retreat with respect to the main body 41 without being connected to the main body 41. An annular finger hooking portion 43 is provided at a proximal end of the main body 41.

The sliding portion 42 has slider 44, a second slider 45, and a third slider 46 that are slidably attached to the main body 41, respectively. An end of the first wire 21 is fixed to the first slider 44. An end of the second wire (not shown) is fixed to the second slider 45. An end of the pulling wire 18 is fixed to the third slider 46. Accordingly, as the respective sliders advance or retreat in the axis direction of the main body 41 with respect to the main body 41, the corresponding wires are advanced or retreated with respect to the base 12 of the main body 41 and the distal part 10, the pusher part 13 and the needle 11 can be operated.

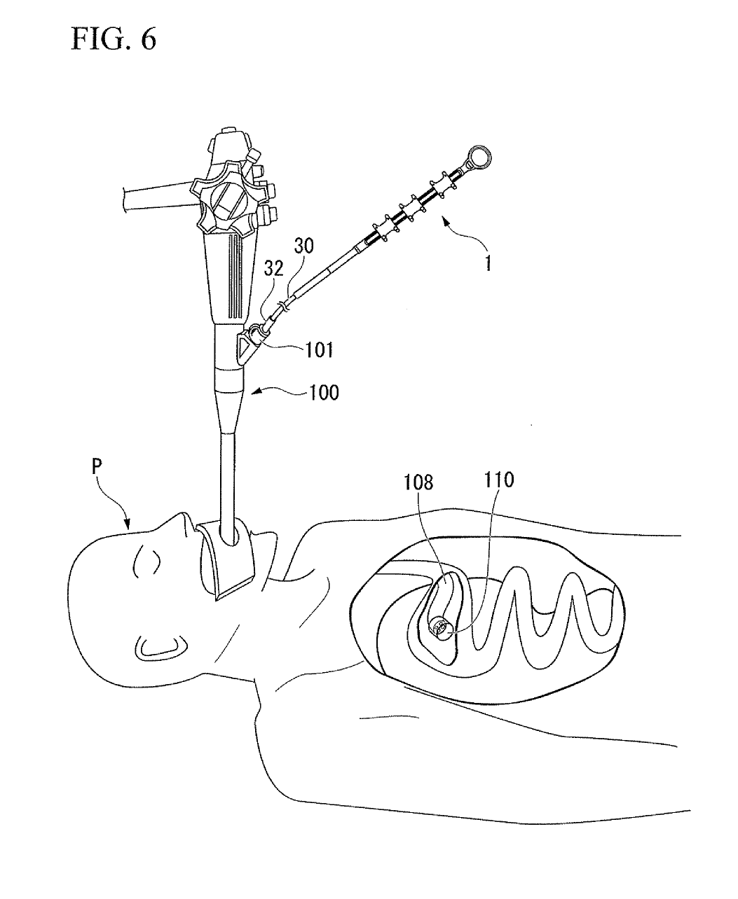

The flow of the suturing method of the invention using the suturing instrument I configured as described above will be described with reference to FIGS. 6 to 17. The suturing instrument 1 is inserted into a body cavity via a general common endoscope 100 including an insertion part 108 that has flexibility as shown in FIG. 6.

First, as shown in FIG. 6, after a cylindrical cap (pressing part) 110 that has transparency is mounted on the distal end of the endoscope 100, the endoscope is inserted into a body cavity of a patient P or the like. Then, the distal end of the endoscope 100 is moved near a target to be treated (hereinafter referred to as a “target tissue”) where a perforation or the like exists. FIG. 6 shows a case where the target tissue is a stomach (a hollow organ).

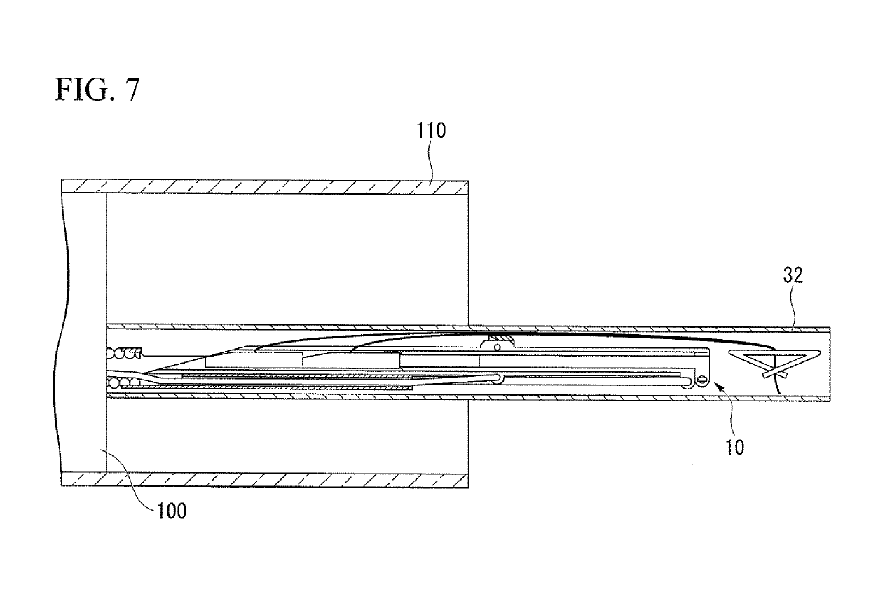

Next, the distal part 10 of the suturing instrument 1 is inserted into a forceps opening 101 of an endoscope 100, and a distal end of the resin sheath 32 is projected from a working channel of the endoscope 100. Here, as shown in FIG. 7, the stopper 105 is loaded into the inside of the resin sheath 32.

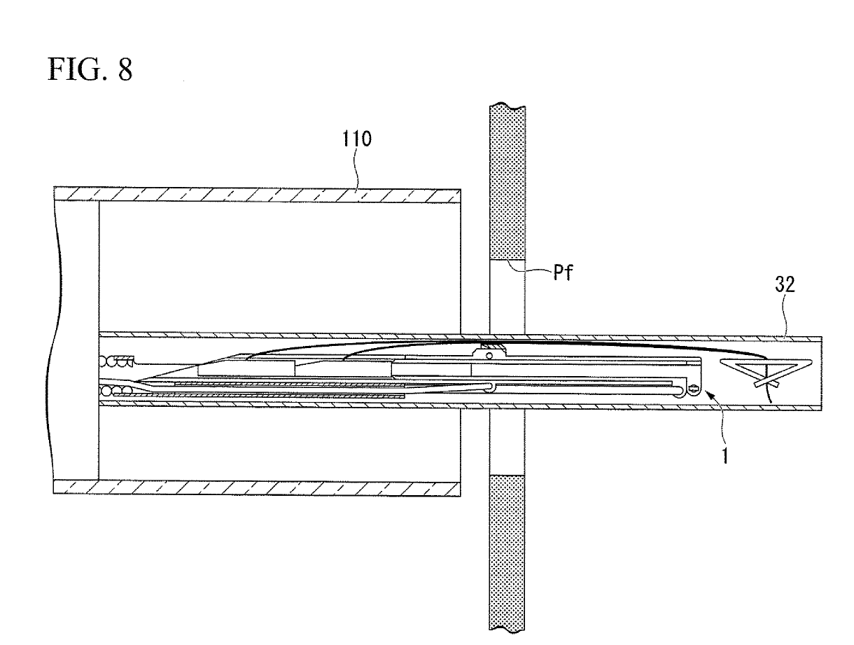

As shown in FIG. 8, an operator projects the suturing instrument 1 accommodated in the resin sheath 32 from the cap 110, and inserts the suturing instrument into a perforation Pf. Thereby, a portion of the distal part 10 of the suturing instrument 1 containing the needle 11 moves to the outside of the stomach.

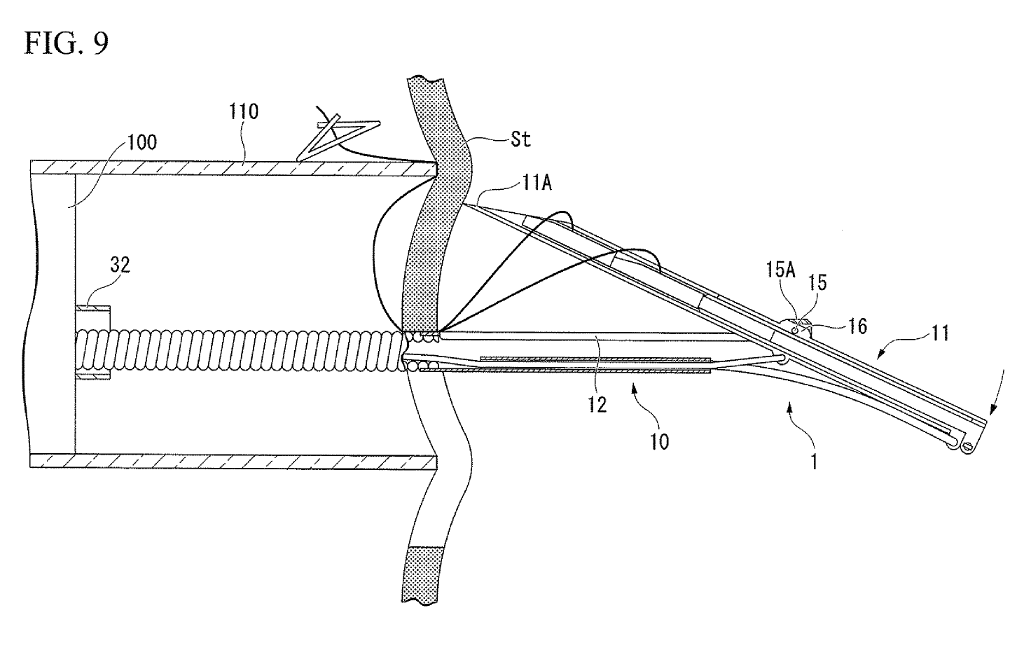

While the suturing instrument 1 is held, the operator retreats the resin sheath 32, and exposes the distal part 10 of the suturing instrument 1. In this state, if the operator pulls the third slider 46 of the operating part 40, the needle 11 is turned around the turning shaft 15, and as shown in FIG. 9, the needle 11 is brought into a second state, and the distal end 11A is directed to the target tissue St. The angle at which the needle 11 forms with the base 12 is held in a steady manner, as the stopper 15A and the pivot portion 16 provided near the turning shaft 15 abut each other. In addition, in the present embodiment, the needle 11 is unfolded after the distal part 10 is inserted into the perforation Pf. Instead of this, however, the needle 11 may be unfolded before the distal part being inserted into the perforation Pf, the coil sheath 31 may be rotated to set the direction of the needle 11 in a suitable direction, and then, the needle 11 may be inserted into the perforation Pf.

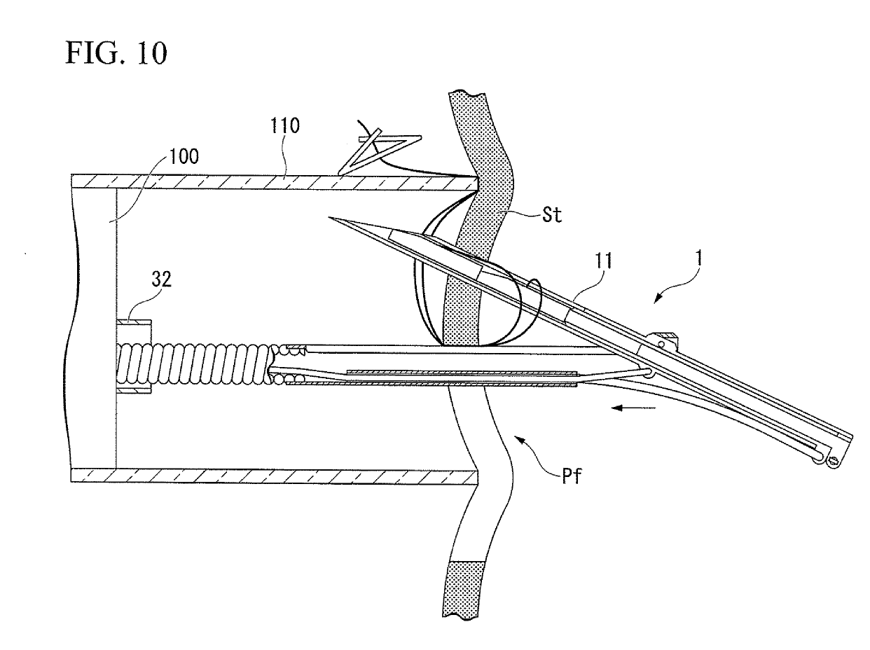

Next, the operator retreats the suturing instrument 1 with respect to the endoscope 100 while advancing the endoscope 100 to press the near side of the target tissue St around the perforation Pf using the cap 110, and as shown in FIG. 10, inserts the needle 11 from the back side of the target tissue St to pass the needle through the target tissue. Since the target tissue St is supported by the cap 110, displacement or deformation of the target tissue St at the time of insertion of the needle 11 is suppressed. As a result, the operator can easily insert the needle 11 into the target tissue St to pass the needle through the target tissue.

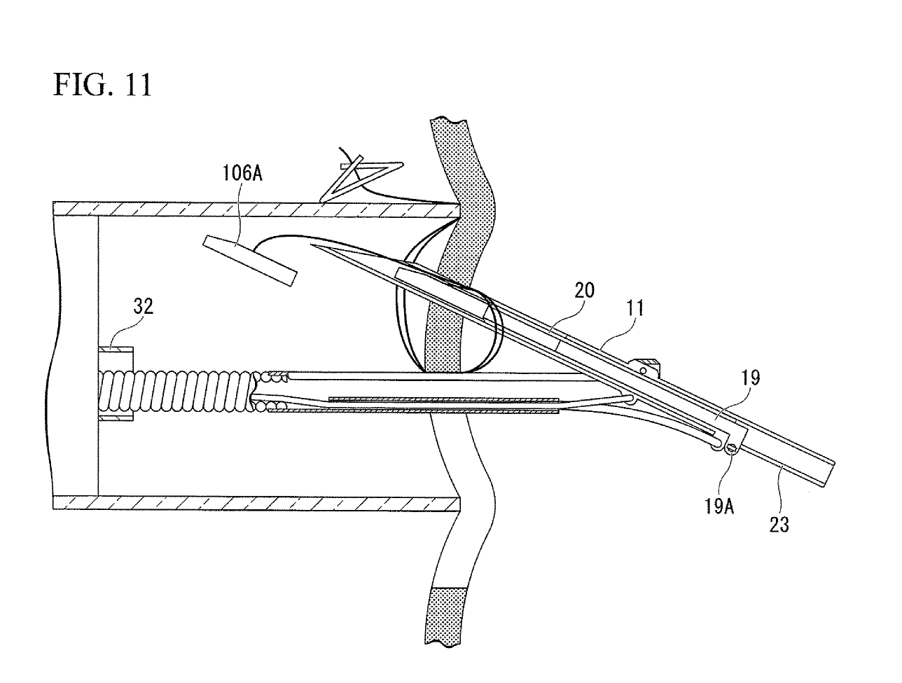

In the place where the needle 11 is passed through, the operator pulls the first slider 44 to the proximal side while holding a state in which the third slider 46 is pulled. Then, the second pusher 20 and the first pusher 19 are integrated, and move toward the distal end 11A in the needle 11. Since the connecting portion 19A of the first pusher 19 cannot advance into the second region 23B of the groove 23, the first pusher 19 stops in a place where the connecting portion 19A has moved to a connection part between the first region 23A and the second region 23B, and the operator cannot pull the first slider 44 anymore. At this time, since the second pusher 20 pushed by the first pusher 19 moves only by an amount corresponding to one anchor in the needle 11, as shown in FIG. 11, only the first anchor 106A is reliably pushed out and ejected from the needle 11.

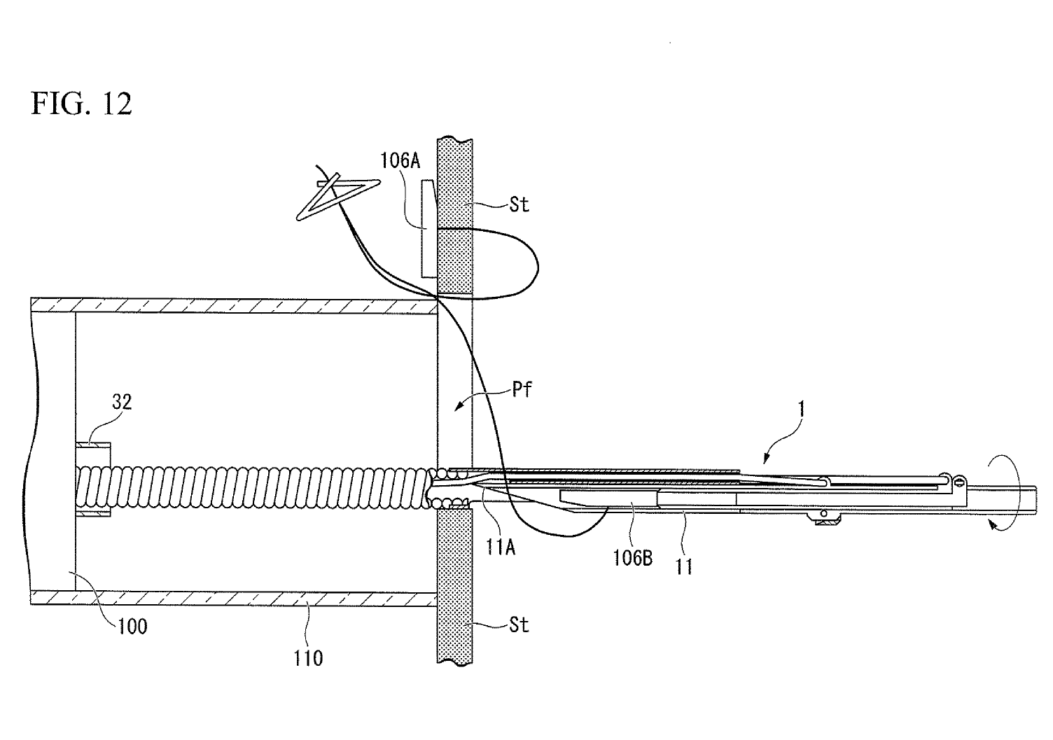

After the ejection of the first anchor 106A, the operator advances the whole suturing instrument 1, and extracts the needle 11 from the target tissue St. Then, the third slider 46 is advanced to bring the needle 11 into the first state, and then, as shown in FIG. 12, the suturing instrument 1 is rotated by approximately 180 degrees around the axis. Further, the endoscope 100 is moved so that the cap 110 can press the periphery of the target tissue St at the position, which faces the part through which the needle 11 has passed, across the perforation Pf.

In addition, although the rotational operation of the suturing instrument 1 described above may be performed while the needle 11 is in the second state, the rotational operation of the suturing instrument while the needle is in the first state is safer because, for example, a situation in which the distal end of the needle 11 touches the target tissue St unintentionally does not easily occur.

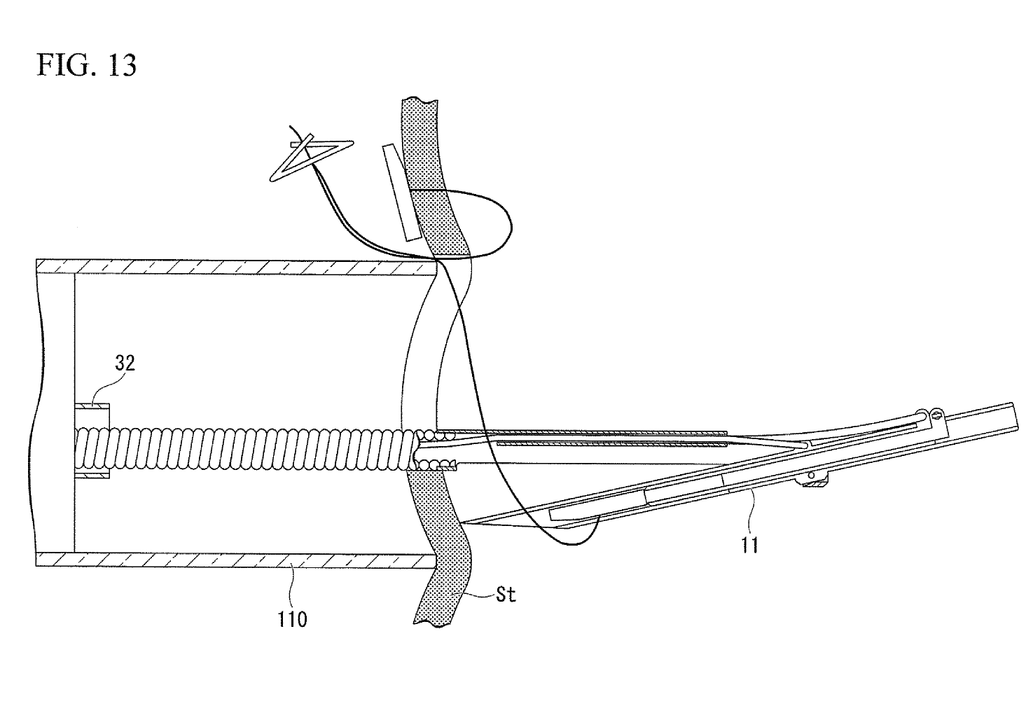

Subsequently, the operator operates the third slider 46 to bring the needle 11 into the second state again as shown in FIG. 13, and penetrates the target tissue St with the needle 11 while pressing the target tissue St from the near side with the cap 110.

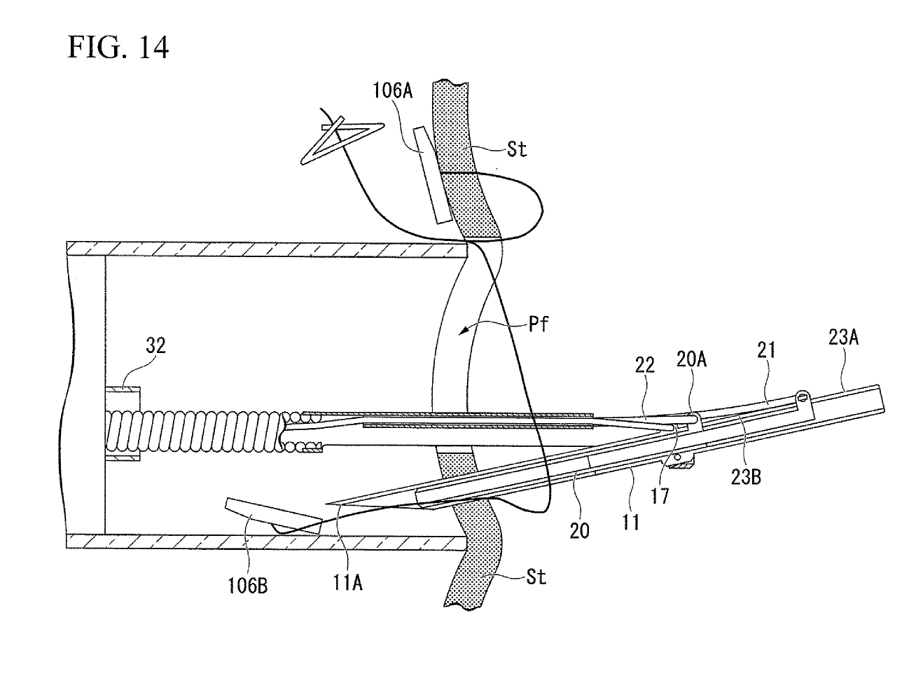

In the place where the needle 11 has penetrated the target tissue St, the operator pulls the second slider 45 to the proximal side. Then, the second pusher 20 pulled by the second wire 22 moves further toward the distal end 11A side in the needle 11 as the connecting portion 20A moves in the second region 23B of the groove 23. As the connecting portion 20A moves up to a terminal point of the groove 23 located in the vicinity of the pulling portion 17, the second pusher 20 moves in the needle 11 by approximately a length equivalent to one anchor. Thereby, as shown in FIG. 14, the second anchor 106B is ejected from the needle 11. By the operation so far, the first anchor 106A and the second anchor 106B are locked to the target tissue St at positions that face each other across the perforation Pf.

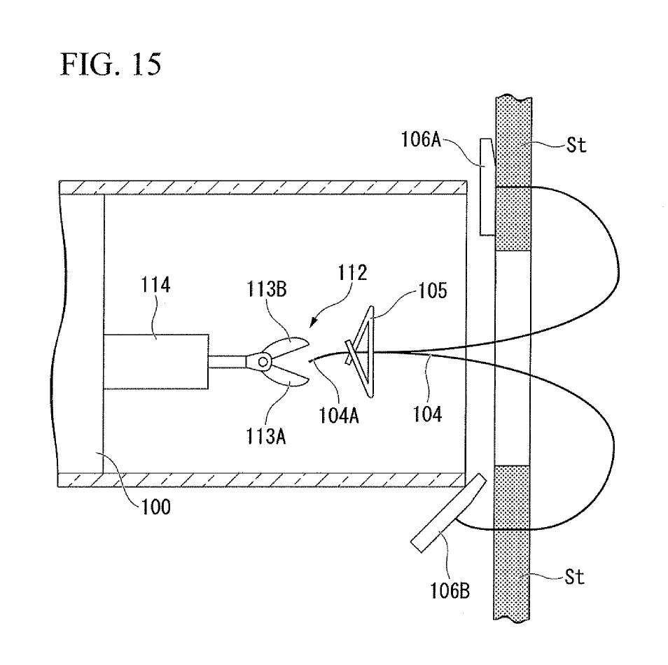

The operator removes the suturing instrument 1 from the endoscope 100, inserts a treatment tool for completing suturing into the forceps opening 101, and as shown in FIG. 15, projects the treatment tool from the endoscope 100. FIG. 15 shows a treatment tool including the forceps 112 that has a pair of forceps pieces 113A and 113B that can be opened and closed as an example of such a treatment tool, and a forceps sheath 114 into which forceps 112 are inserted so as to be able to advance or retreat.

The operator advances the forceps sheath 114 with respect to the forceps 112, with the vicinity of the middle point 104A of the suture thread 104 being gripped by the forceps 112. Then, while the stopper 105 and the forceps sheath 114 abut each other, only the suture thread 104 is accommodated in the forceps sheath 114, and the distance between the stopper 105 and each of the anchors 106A and 106B becomes short.

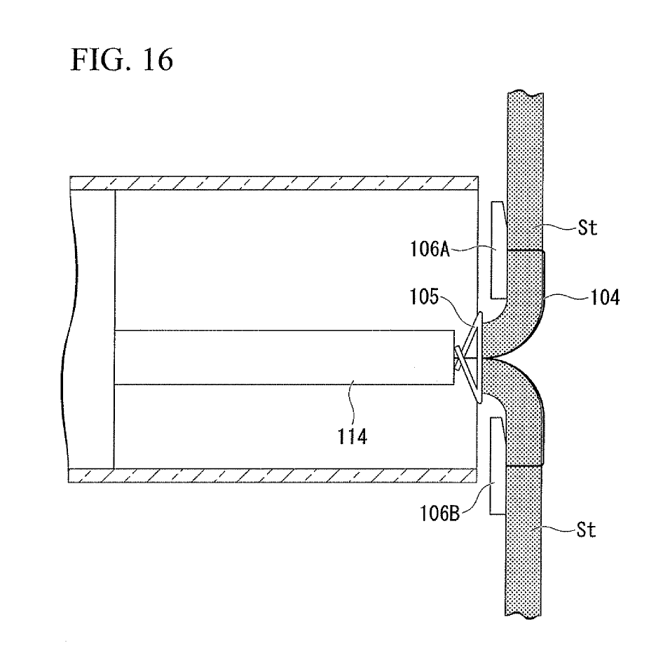

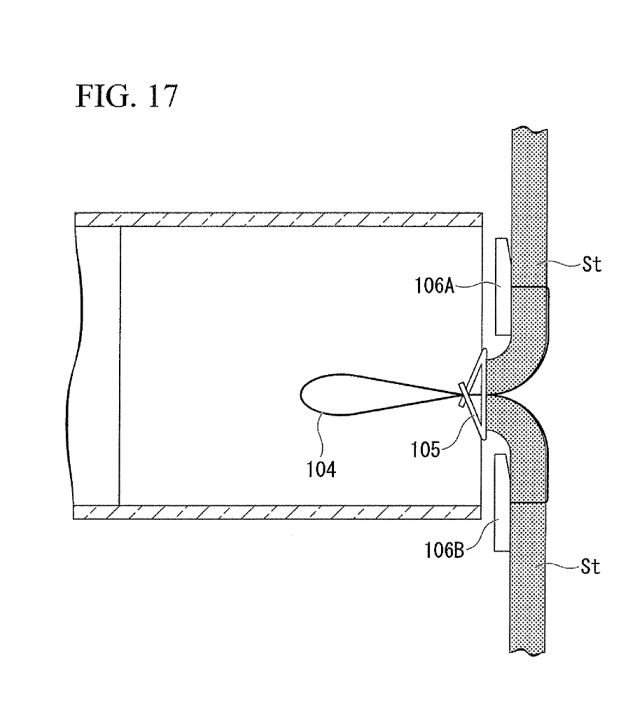

Since the anchors 106A and 106B are locked to the target tissue St, respectively, as shown in FIG. 16, the stopper 105 and the respective anchors 106A and 106B are brought close to each other, and the target tissue St is pulled toward the stopper 105 along with the respective anchors 106A and 106B. In this way, the target tissue St is brought into close contact with the perforation Pf so as to block the perforation, and as shown in FIG. 17, is sutured, whereby the series of treatments ends.

In the suturing method of the present embodiment, by operating the third slider 46 of the suturing instrument 1, the posture of the needle 11 can be switched to the second state as shown in FIG. 9, and the needle can be inserted toward the near side where the target tissue can be observed using the endoscope 100, from the back side of the target tissue St. Accordingly, by visually recognizing the distal end of the needle 11 to be inserted into a target tissue, puncture can be performed safely, and the anchors 106A and 106B of the suturing unit 103 can be ejected reliably.

Additionally, since the pusher part 13 is provided at the distal part 10, the anchors can be more reliably ejected by the pusher part 13 by operating the operating part 40 to pull the first wire 21 and the second wire 22.

Generally, in order to eject two anchors using a pusher, a stroke (travel distance) equivalent to the length of two anchors is needed. Accordingly, a suturing instrument with such a structure, the range (hard length) where a high-rigidity member is arranged in the distal part becomes the sum of the length of the needle 11 and the stroke of the pusher, and the length becomes the sum of the length of the needle 11 and a length equivalent to two anchors at the minimum.

However, in the suturing instrument 1 of the present embodiment, the needle 11 is provided with the groove 23 where the respective pushers 19 and 20 are movable. Thus, even in a state where the respective pushers 19 and 20 are completely accommodated in the needle 11, the pushers can be moved toward the distal end 11A by pulling the first wire 21 or the second wire 22. Accordingly, in order to secure a stroke, the projecting length from the needle can be shortened. As a result, it is possible to provide a suturing instrument in which the hard length is kept from becoming long and the insertion property to the endoscope 100 is held appropriately.

Moreover, since the pusher part 13 has two pushers of the first pusher 19 and the second pusher 20, and the number of the anchors and the number of the pushers are the same, the two anchors can be reliably ejected one by one. Moreover, since the groove 23 has the first region 23A and the second region 23B, the respective anchor 106A and 106B can be reliably ejected one by one by an easy operation of pulling the first slider 44 to a limit and then pulling the second slider 45 to a limit.

Moreover, since the pivot portion 16 of the needle 11 is located on the outer peripheral surface of the same phase as the part of the slope of the distal end 11A closest to the proximal side, the sharp distal end 11A does not easily protrude from the base 12 in the first state. Accordingly, the distal end 11A does not easily interfere with the inner surface or the like of the working channel of the endoscope or the like, for example, when the suturing instrument 1 is removed. In addition, even in a case where the needle 11 is slightly turning from the first state, as the inner surface or the like of the channel and the slope contact each other at the time of removal, the needle 11 easily returns to the first state. As a result, it is possible to provide a suturing instrument that can perform removal or the like smoothly and that is easier to use.

Additionally, according to the suturing method of the invention, when the needle 11 is inserted toward the near side of the target tissue St from the back side thereof, the insertion is performed while pressing the target tissue St from the near side thereof, using the cylindrical cap 110 attached to the distal end of the endoscope 100.

Since many of hollow organs that are targets of the present suturing method are excellent in extensibility, even if the needle is pushed against a hollow organ from one direction, tissue merely stretches or moves and may not be easily punctured. However, in the present suturing method, the target tissue St is punctured while being pressed toward the needle 11 by the cap 110. Thus, the needle can be inserted in a state where the target tissue has stretched to some extent, thereby preventing the situation as described above to make it possible to perform puncturing appropriately.

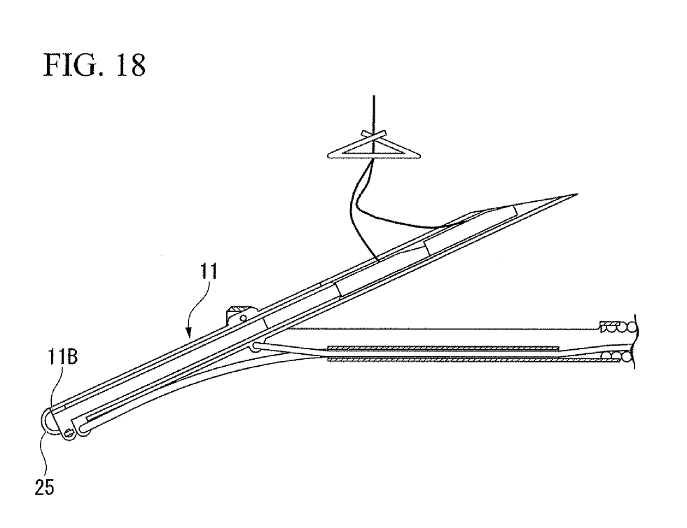

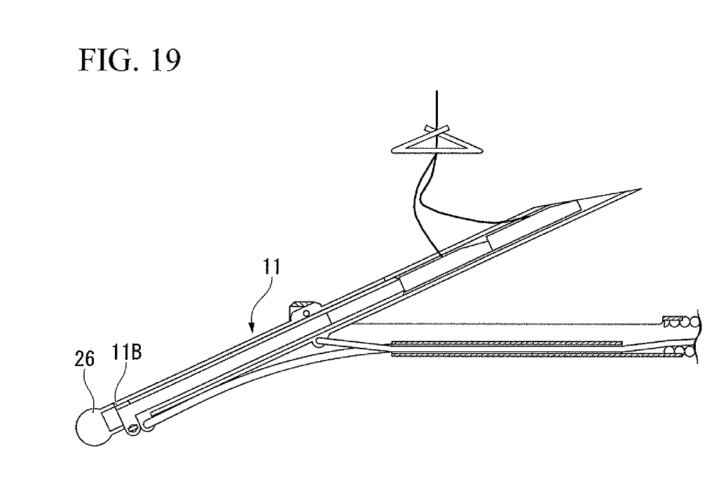

In the suturing instrument 1 of the present embodiment, when the needle 11 is in the first state, the proximal end 11B of the needle 11 is located at the distal end of the suturing instrument 1. Therefore, as in a modification shown in FIG. 18, a cap-shaped protective member 25 that has a curved surface is attached to the proximal end 11B. Thereby, even in a case where the proximal end 11B has contacted a target tissue, a surrounding tissue, or the like unintentionally, the tissue can be prevented from being damaged. The protective member may not be the cap-shaped protective member, for example, may be a substantially spherical protective member 26 or a true spherical protective member (not shown)), as in a modification shown in shown in FIG. 19.

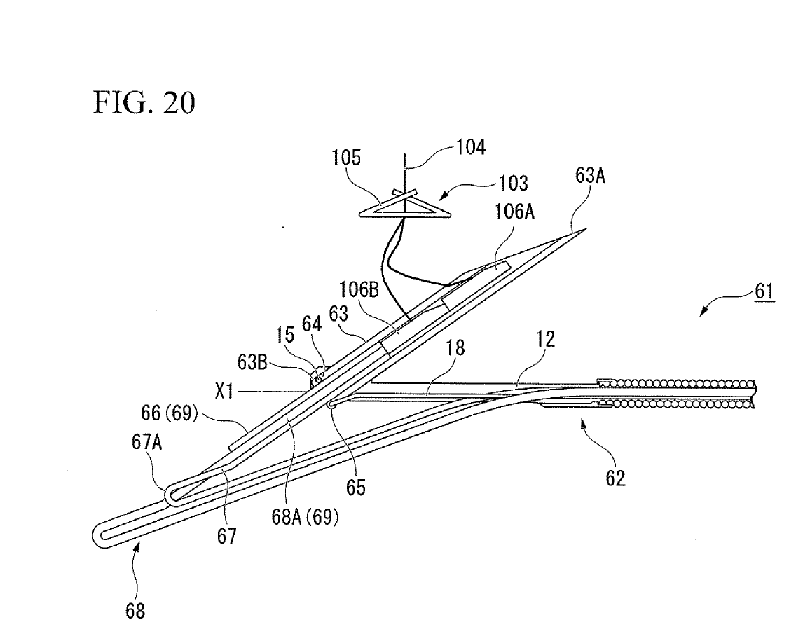

Next, a second embodiment of the invention will be described with reference to FIG. 20. A difference between the suturing instrument 61 according to the present embodiment and the suturing instrument 1 according to the first embodiment is the configuration of the distal part. In addition, in the following description, the same components as those that have already been described will be denoted by the same reference numerals, and duplicate descriptions will be omitted.

FIG. 20 is a partially enlarged view showing a distal part 62 of the suturing instrument 61. A needle 63 is shorter than the needle 11, and a pivot portion 64 and a pulling portion 65 are provided near a proximal end 63B. The groove 23 is not provided in the needle 63.

The second pusher 66 is tubularly formed of materials, such as metal, and the second wire 67 is connected to a proximal end that protrudes from the needle 63 by welding or the like. A distal part of the second wire 67 connected to the second pusher 66 is folded back in the shape of a hairpin, and a part ahead of a folded-back point 67A and a part behind a folded-back point 67A become substantially parallel to each other.

The first wire 68, similarly to the second wire, is folded back in the shape of a hairpin, and a distal part 68A that is bent after being folded back is inserted through the second pusher 66, and advances into the needle 63. That is, the distal part 68A inserted into the needle 63 functions as the first pusher.

Although the usage of the suturing instrument 61 configured as described above is almost the same as that of the suturing instrument 1, if the third slider 46 is pulled to bring the needle 63 into the second state, as shown in FIG. 20, the first wire 68 and the second wire 67 are curved to the proximal side of the needle 63. A cutout that is formed in an outer peripheral surface of the base 12 in a phase that faces the turning shaft 15 across the axis X1 so as not to interfere with curved portion extends longer in the axis direction X1 than that of the suturing instrument 1. It should be noted that this suturing instrument is the same as the suturing instrument 1 in that the outer peripheral surface of the phase is left behind to such a degree that a distal end 63A of the needle 63 can be supported.

If the first wire 68 is advanced until the distal part 68A of the first wire 68 is mostly accommodated in the second pusher 66 when the anchors are ejected, the distal part 68A as the first pusher advances by one anchor, and the first anchor 106A is ejected first. Subsequently, if the second wire 67 is pulled, the second anchor 106B is ejected by the second pusher 66.

When the first wire 67 and the second wire 66 are drawn, since the advance direction of the pushers differs from the retreat direction of the wires, a force that mutually separates a distal end and a proximal end in each wire acts. However, since each wire is folded back in the shape of a hairpin, this folded-back part holds the shape of each wire against the force that mutually separates the distal end and the proximal end. As a result, the pushers can be appropriately advanced within the needle.

In the suturing method of the present embodiment, the suturing instrument 61 includes a pusher part 69 made up of the distal part 68A and the second pusher 66. Thus, the respective anchors 106A and 106B of the suturing unit 103 can be reliably ejected similarly to the suturing instrument 1.

In the present embodiment, the first wire 68 and the second wire 67 are bent in the shape of a hairpin, and the distal part 68A of the first wire 68 functions as the first pusher. Thus, it is preferable that single wires with relatively high rigidity be used as these wires.

Although the preferred embodiments of the invention have been described above, it should be understood that the invention is not limited to these embodiments. Additions, omissions, substitutions, and other modifications can be made without departing from the scope of the present invention.

For example, during the insertion of the needle, it is necessary that the third slider is held in a pulled state. Therefore, the third slider may be adapted to be capable of being temporarily fixed to the main body of the operating part by a screw, a ratchet mechanism, or the like.

Additionally, the number of the anchors in the suturing unit is not limited to two, and may be arbitrary as long as a plurality of anchors is provided. In this case, it is preferable that the same number of pushers as the number of the anchors being provided, since the anchors can be easily ejected one by one, this is preferable.

Moreover, for example, in a case where a target tissue has sufficient thickness or the like, an anchor may be ejected into the tissue, with the distal end of the needle being located in the tissue.

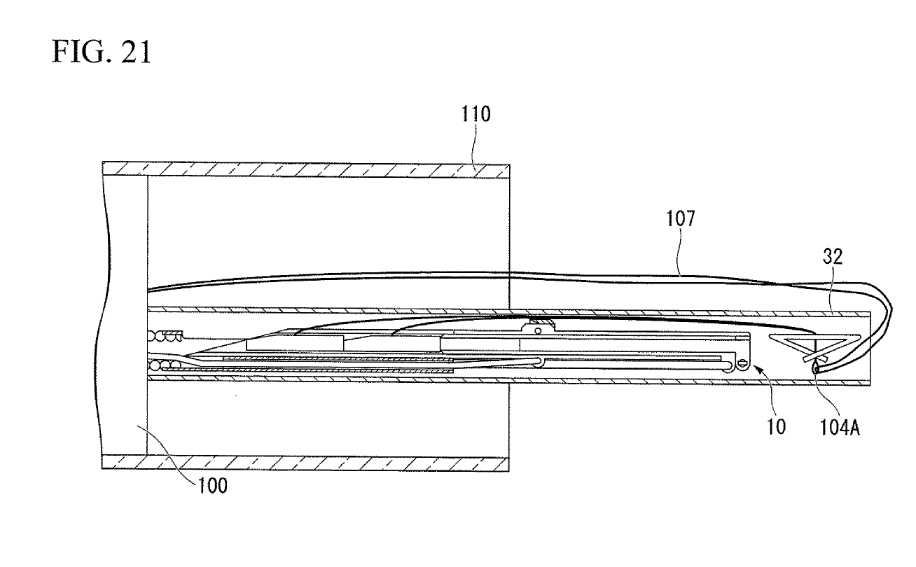

Additionally, as shown in FIG. 21, a pulling thread 107 may be passed through and engaged with the vicinity of a middle point 104A of a thread of the suturing unit 103, ends of the pulling thread 107 may be drawn out of the body from the forceps opening 101 of the endoscope 100, and the operator may pull the thread 107 so that the middle point 104A of the thread can be pulled. By so doing, at the time of tight binding of the suturing unit 103 shown in FIGS. 15 to 18, it is possible to prevent cases where the stopper 105 and the middle point 104A deviate to the outside of a body cavity or the like from the perforation Pf, and the middle point 104A is no longer held by the forceps 122. In addition, the pulling thread 107 is removed to the outside of the body from the forceps opening 101 by pulling one end after the end of treatment. Additionally, in a case where the ends of the pulling thread 107 locked to the middle point 104A are bound in the shape of a loop, one end may be pulled after the loop is cut at one place.

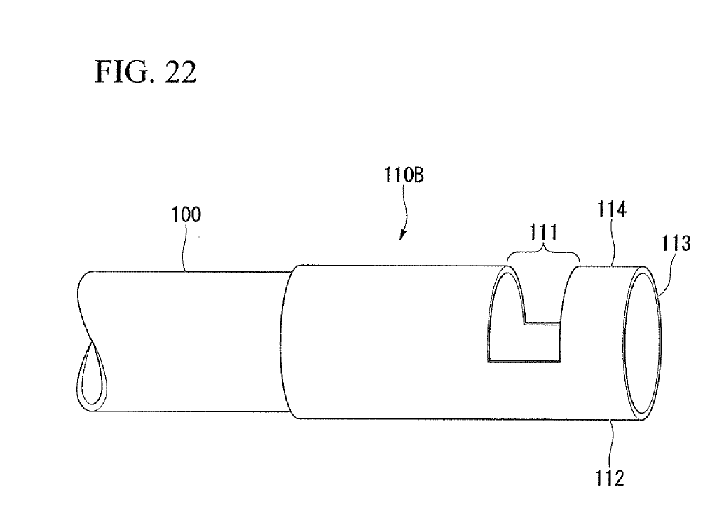

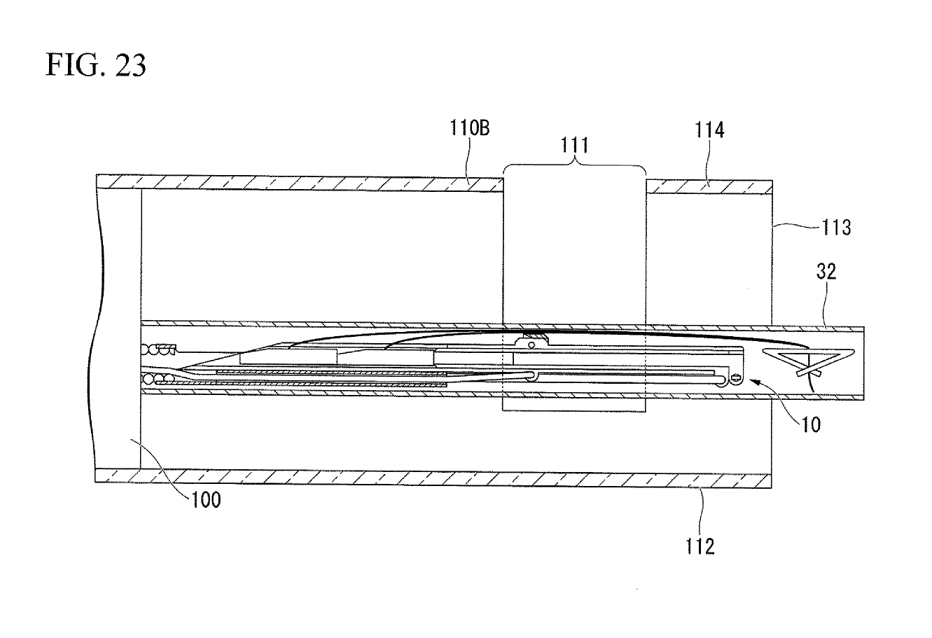

Additionally, the cap used for the suturing method of the invention does not necessarily have to be a cylinder. For example, as in a cap 110B shown in FIGS. 22 and 23, a first slit 111 may be formed that extends in the circumferential direction of a substantially cylindrical cap and communicates with an inner cavity and that has such a width (a dimension in the axis direction of the cap) that the target tissue St can advance.

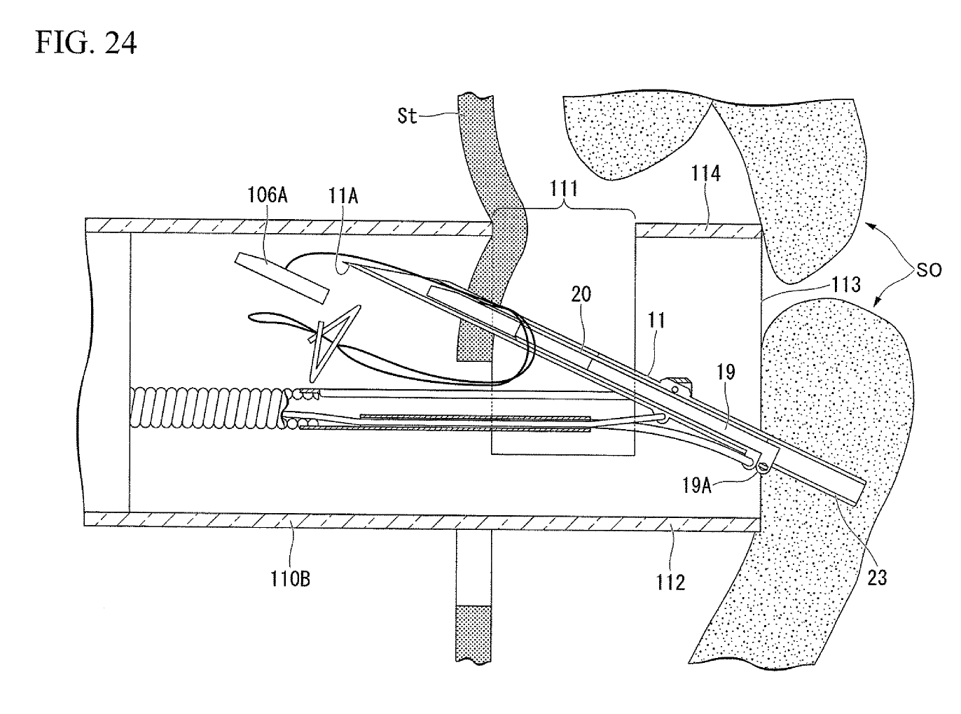

As shown in FIG. 24, depending on parts of the target tissue St, surrounding organs SO, such as the small intestine and the large intestine, may exist outside. By using the endoscope 100 mounted with the cap 110B, and locating the target tissue St in an inner cavity of the cap 110B through the first slit 111, the surrounding organs SO do not enter the first slit 111, and a target organ can be distinguished from other surrounding organs. At this time, the surrounding organs SO are excluded under pressure by a side wall 112 and a distal end 113 of the cap 110B, and a gap is formed between the target organ St and the surrounding organs SO by a separating portion 114 formed by a side wall between the first slit 111 and the distal end 113, and the distal end 113.

By operating the needle 11 so that the sharp distal end 11A of the needle 11 does not protrude to the outside of the cap 110B from the separating portion, and by puncturing the target tissue St located in the cavity of the cap 110B, it is possible to reliably puncture only the target tissue St without damaging surrounding organs with the needle.

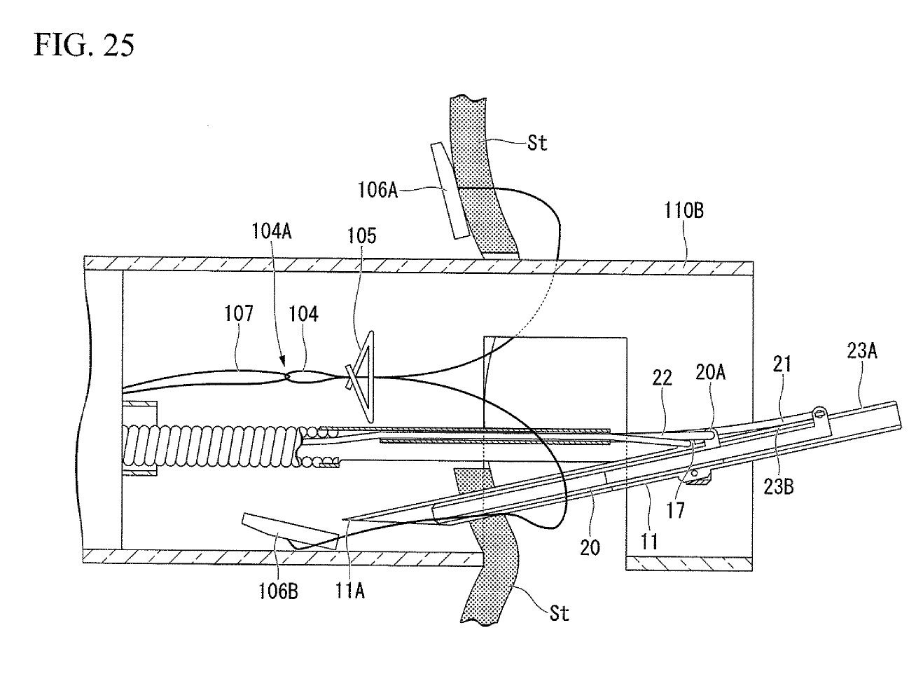

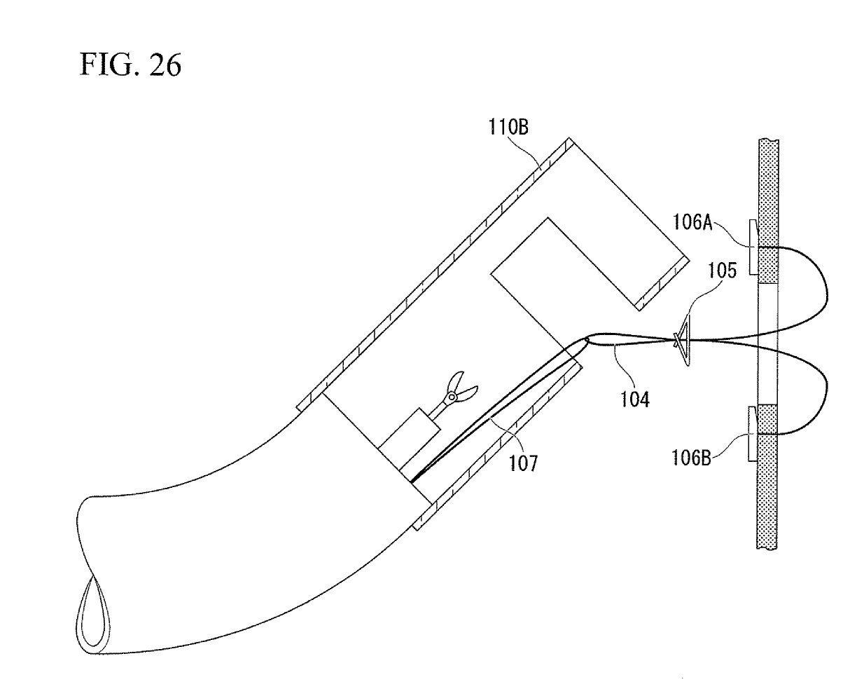

Here, as shown in FIG. 25, in a case where the pulling thread 107 is passed through the middle point 104A of the suture thread of the suturing unit 103, as shown in FIG. 26, when a plurality of anchors 106A and 106B is placed in the target tissue St and tightly bound by the stopper 105, the suture thread 104 and pulling thread 107 pass through the first slit 111. Therefore, tight binding of the suture thread 104 becomes difficult.

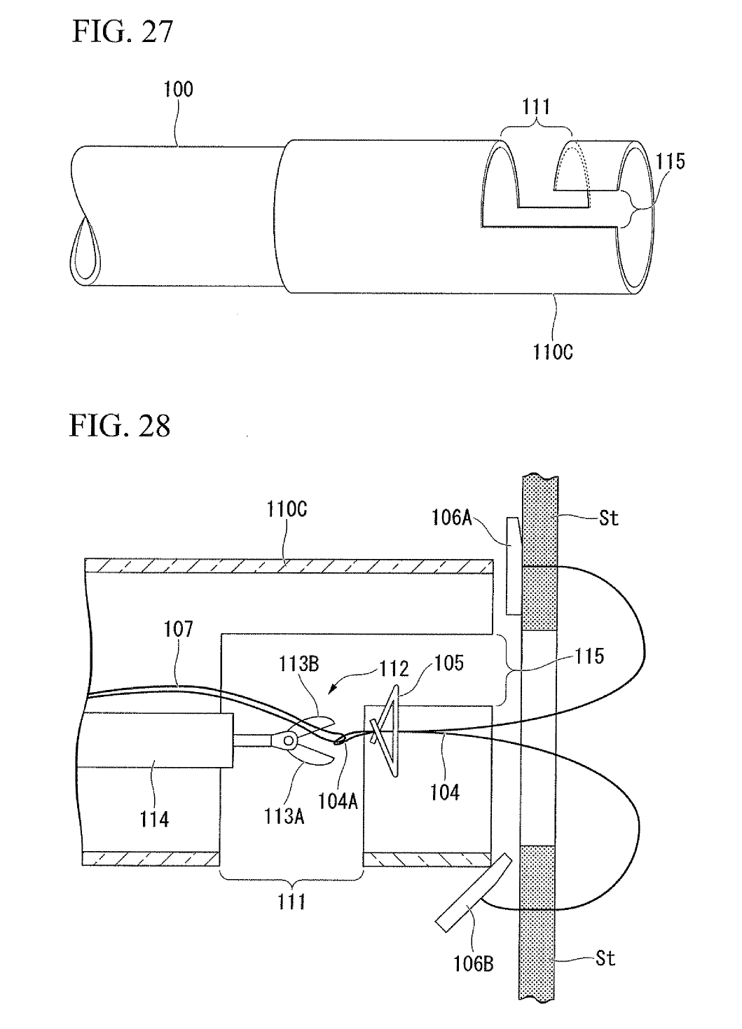

Thus, as shown in FIGS. 27 and 28, a cap 110C having a second slit 115 that communicates with the first slit 111 and that extends parallel to the axis of the cap and opens to the distal end may be used. In this case, by passing the suture thread 104 and pulling thread 107 through the second slit 115 after the anchors 106A and 106B are placed in the target tissue St, the suture thread 104 and the stopper 105 can be located at the front with respect to the distal end surface of the endoscope, and a tight-binding operation of the suturing unit 103 can be performed easily.

1. A suturing method of tissue using a suturing apparatus including a suturing instrument that has a hollow anchor holder at a distal part thereof, the hollow anchor holder having a sharp distal end and having a plurality of anchors accommodated in an inner cavity thereof, and that performs suturing of tissue using a suturing unit in which the plurality of anchors is attached to ends of a suture thread; an endoscope including an insertion part having a channel that allows the suturing instrument to be inserted thereinto; and a pressing part capable of being mounted on a distal end of the endoscope, the suturing method comprising:
inserting the endoscope having the pressing part attached to the distal end thereof into a body cavity, and moving the endoscope to a near side of the tissue;
inserting the suturing instrument having the suturing unit mounted thereon into the channel, and projecting the distal part from the distal end of the endoscope;
moving the anchor holder having the plurality of anchors accommodated therein to a back side opposite to the near side of the tissue; and
inserting the distal end of the anchor holder into the tissue from the back side opposite to the near side while the tissue is pressed by the pressing part from the near side, and ejecting at least one of the plurality of anchors from the anchor holder.

2. The suturing method of tissue according to claim 1,
wherein the distal part has a base provided with a turning shaft having the anchor holder turnably pivoted thereon,
the anchor holder is pivoted on the turning shaft in a state where the distal end thereof is directed to a proximal side of the base, and
the anchor holder is inserted into the tissue in a state where the distal end forms an angle with the base so as to separate from an axis of the base.
